(19) United States
(12) Patent Application Publication
(10) Pub. No.: US 2007/0243608 A1
(43) Pub. Date: Oct. 18, 2007

(54) Platelet bioreactor

(75) Inventors: Michael Kyba, Plano, TX (US); Michelina Iacovino, Dallas, TX (US)

Correspondence Address: RICHARD ARON OSMAN, 4070 CALLE ISABELLA, SAN CLEMEMTE, CA, 92672, US

(73) Assignee: Board of Regents, The University of Texas System

(21) Appl. No.: 11/404,213

(22) Filed: Apr. 14, 2006

(51) Int. Cl. C12N 5/08; C12N 5/06; C12N 15/867

(52) U.S. Cl. 435/325; 435/372; 435/456

(57) ABSTRACT

Platelets are produced in a bioreactor in which hematopoietic stem cells (HSCs) are cultured to produce megakaryoctye progenitors, megakaryocytes, and platelets. The HSCs are stably genetically engineered to express HoxA2 or HoxB2 which induces HSC proliferation and promotes generation of megakaryocytes.

### BACKGROUND OF THE INVENTION

The field of the invention is a bioreactor and methods for the generation of platelets in vitro from cultured stem cells and stem cell progeny genetically engineered to express HoxA2 or HoxB2.

Hox genes have been studied for their roles in embryonic development, and HoxA2 has been shown to be involved in, inter alia, skeletogenesis (Creuzet, 2005), hindbrain development (Eddison, 2004), and differentiation of proximal mesenchymal derivatives and vasculogenesis in the lung (Cardoso, 1996). Neuronal defects in the hindbrain of HoxA1, HoxB1 and HoxB2 mutant mice reflect regulatory interactions among these Hox genes (Gavalas, 2003). Hoxa2 expressed by retroviral vectors in the anterior-most hindbrain of developing chick embryos led to the generation of motor neurons in this region which is normally devoid of this cell type (Jungbluth, 1999).

Hox genes are arranged in clusters. An ancestral cluster has been multiply duplicated to give four clusters in mammals. Thus, up to four paralogs exist for any Hox gene (for example, HoxA1, HoxB1, HoxC1, and HoxD1 comprise paralog group one, which contains a full complement of four members). Paralog group 2 has two members: HoxA2 and HoxB2. Profound conservation of function exists within a paralog group such that the protein-coding sequences of Hox genes are phenotypically interchangeable (Greer, 2000).

At least 16 of the 39 Hox genes, including HoxA2 and HoxB2, are normally expressed in CD34+ human marrow cells (Sauvageau, 1994). Giampaolo et al (1994) reported that HoxB2 is transiently expressed at low level in the granulopoietic pathway, and is detected only in terminal stages of erythropoiesis. Several Hox genes have been over-expressed in hematopoietic cell lines, cultured ES cells, and bone marrow cells to study their roles in hematopoiesis (Lawrence, 1996); however, very few Hox genes have an established function in hematopoiesis (Shivdasani, 2001).

Thorsteinsdottir et al (1997) reported that colonies generated in vitro from HoxA10-transduced murine bone marrow cells (but not from HoxB4-transduced cells) presented a unique large colony type containing megakaryocytes and blast cells. Furthermore, “[t]he generation of this unique colony type in HOXA10 cultures was accompanied by a proportional reduction in multilineage GEMM, granulocyte-macrophage, and granulocyte colonies; moreover, no unilineage macrophage colonies could be detected among [HOXA10-transduced] colonies.” Id. 497. This unique colony was also generated in vitro from cells posttransplantation (cells extracted from the bone marrow of irradiated mice transplanted with the HoxA10-transduced bone marrow cells); however, “[c]uriously, despite this high frequency in bone marrow of HOXA10 mice of a progenitor cell with potential to differentiate in vitro into megakaryocytes, inspection of bone marrow and peripheral blood smears from HOXA10 mice revealed no gross increase in megakaryocyte or platelet counts.” (Id. 500).

The authors suggested that “under normal physiological conditions, HOXA10 might be involved in processes of hematopoietic lineage commitment and differentiation, playing a positive role in megakaryopoiesis but negatively regulating monocytic and B-cell development. These results add to the recognition of Hox genes as important regulators of hematopoiesis and point to Hox gene-specific effects that likely reflect their regulation of different target genes during hematopoietic development.” Id. p. 504. This publication does not mention HoxA2 or HoxB2, and does not suggest that overexpression of any Hox gene, HoxA10 included, could be used to promote production of normal megakaryocytes.

### BRIEF SUMMARY OF THE INVENTION

The invention provides a platelet-producing bioreactor comprising a culture vessel containing (a) hematopoietic stem cells (HSCs) stably genetically engineered to express HoxA2 or HoxB2, and (b) platelet-producing megakaryocyte progeny of the HSCs. In various embodiments, the HSCs are derived from a stem cell source selected from cord blood, bone marrow, embryonic stem cells differentiated in vitro, and an immortalized hematopoietic stem cell line cell. In preferred embodiments the HSCs are stably genetically engineered to inducibly express the HoxA2 or HoxB2. In specific embodiments the HSCs are engineered with a self-inactivating lentiviral vector that expresses a HoxA2 or HoxB2 transgene. In a preferred embodiment, HSCs are the progeny of embryonic stem (ES) cells stably genetically engineered to inducibly express HoxA2 or HoxB2, and the culture vessel additionally contains a hematopoietic growth medium containing a reagent that induces expression of the HoxA2 or HoxB2.

In various embodiments, the bioreactor comprises an insoluble matrix which retains the HSCs and/or is in fluid connection with an apheresis device operative to selectively remove platelets from the bioreactor.

The invention also provides methods of making platelets in the bioreactor comprising the step of: culturing the HSCs and the megakaryocyte progeny in the bioreactor to produce the platelets. The method further comprises the step of removing the platelets from the bioreactor. In a specific embodiment, the culturing step comprises continually passaging the HSCs for at least 30 days.

Another aspect of the invention is a method of making platelets, the method comprising the step of: culturing hematopoietic stem cells (HSCs) to produce megakaryocyte progenitors, megakaryocytes, and platelets, wherein the HSCs are stably genetically engineered to express HoxA2 or HoxB2, and wherein the HSCs express the HoxA2 or HoxB2 during the culturing step. In various embodiments, the HSCs are engineered with a self-inactivating lentiviral vector that expresses a HoxA2 or HoxB2 transgene. The method may comprise the further step of purifying the platelets. In various embodiments, the HSCs are derived from embryonic stem (ES) cells, and the method comprises the prior steps of: stably genetically engineering ES cells to inducibly express HoxA2 or HoxB2; and differentiating the ES cells to form the HSCs.

In one embodiment of the method, the culturing step comprises proliferating the HSCs in a hematopoietic growth medium that induces the HSCs to express the HoxA2 or HoxB2 and produce the megakaryocyte progenitors; and passaging the megakaryocyte progenitors to a differentiation medium that does not induce expression of the HoxA2 or HoxB2, wherein the megakaryocyte progenitors differentiate into the megakaryocytes and platelets. The culturing step may comprise continually passaging the HSCs for at least 30 days without exhaustion of the HSCs.

### DETAILED DESCRIPTION OF THE INVENTION

We have discovered that expression of HoxA2 and/or HoxB2 in hematopoietic stem cells promotes self-renewal and proliferation of the stem cells and preferentially generates large numbers of megakaryocyte progenitor cells to the exclusion of other lineages. The invention provides stem cells capable of hematopoiesis that have been genetically engineered to express HoxA2 or HoxB2. The engineered stem cells can be cultured to yield megakaryocyte progenitors that differentiate into megakaryocytes that produce platelets. In a specific embodiment, the invention provides a platelet-producing bioreactor comprising a culture vessel that contains HoxA2 or HoxB2-expressing hematopoietic stem cells (HSCs) and platelet-producing megakaryocyte progeny of the HSCs. Platelets generated by the bioreactor are suitable and sufficient for use in transfusion therapy; in preferred embodiments, the bioreactor provides platelets suitable and sufficient for repeated transfusion therapy provisions over long-term (e.g. at least a week, month or year).

In one aspect, the invention provides a method of making platelets comprising culturing hematopoietic stem cells (HSCs) that are stably genetically engineered to express HoxA2 or HoxB2 under conditions wherein the HSCs produce megakaryocyte progenitors. The megakaryocyte progenitors differentiate to produce megakaryocytes and platelets, and the platelets are purified from the culture.

The HSCs are stably genetically engineered to express HoxA2 or HoxB2 such that the genetic modification is inherited by the progeny of the HSCs. The genetic engineering may be effected at the HSC or an ancestral cell thereof. In one method, genetically modified stem cells are prepared by transfecting stem cells with vectors that contain a HoxA2 or HoxB2 transgene, and selecting for transfected cells. HSCs targeted for transfection can be derived from any suitable stem cell source (e.g. cord blood, bone marrow, immortalized hematopoietic stem cell lines, etc.) The engineered HSCs may also be the progeny of a more primitive stem cell having hematopoietic potential that was transfected to carry the transgene and then differentiated to generate the HSCs. Example of primitive stem cells having hematopoietic potential include embryonic stem (ES) cells and neural stem cells (see e.g. Bjornson, 1999) etc. In a preferred embodiment, the stem cell that is transfected is a mammalian ES cell, preferably a mouse or huinan ES cell.

In specific embodiments, the stem cells are transfected with a viral vector comprising a recombinant HoxA2 or HoxB2 gene. Suitable viral vectors include retroviral vectors (see e.g. Kyba, 2002; Kyba, 2003; and Fischbach, 2005), lentiviral vectors (see e.g. Logan, 2002; Markusic, 2005; Ma, 2003; Ramezani, 2003; Lois, 2002; Barde, 2006; and Suter, 2006) and adenoviral vectors (see e.g. Brun, 2003). In a specific embodiment, the stem cells are human ES cells or HSCs that have been engineered with self-inactivating lentiviral vectors to express the HoxA2 or HoxB2 transgene. The lentiviral vector may contain one or more elements that maintain transgene expression levels after prolonged culture, such as a scaffold attachment region (SAR) (Ma, 2003), the woodchuck hepatitis virus posttranscriptional regulatory element (WRE) (Lois, 2002), and the 5′ HS4 insulator (Ramezani, 2003). Expression of the HoxA2 or HoxB2 transgene may be under the control of any promoter that drives transgene expression in stem cells. Exemplary such promoters include the human elongation factor 1α (EF1α) promoter (Ramezani, 2003), the phosphoglycerokinase promoter (Kyba, 2002), the human ubiquitin-C promoter (Lois, 2002), the HLA-DRalpha promoter (Yu, 2003), and the LTR promoter of the virus itself (Kyba, 2002).

We have found that more platelets are generated if the transgene expression is induced while culturing the HSCs and megakaryocytes in a hematopoietic growth medium in which the HSCs proliferate, and then turned off to promote differentiation of megakaryocytes into platelets. Accordingly, in preferred embodiments, the stem cells are stably genetically engineered to inducibly express HoxA2 and/or HoxB2. Examples of inducible expression systems include Tet-on (e.g. Markusic, 2005; and Barde, 2006), Tet-off (Blesch, 2005), estrogen receptor/transgene fusion systems (e.g. Janes, 2004), etc.

In a preferred embodiment human or mouse ES cells are stably genetically engineered to inducibly express HoxA2 or HoxB2. The ES cells can be proliferated using conventional methods; for example, mouse ES cells are typically proliferated by co-culture on irradiated mouse fibroblasts or on gelatinized culture plates with Leukemia Inhibitory Factor (LIF). Conditions for expanding human ES cells include culture on irradiated murine embryonic fibroblasts (MEF) (e.g. Thomson, 1998) or culture in defined, feeder cell-independent medium supplemented with high concentrations of basic fibroblast growth factor (bFGF) (e.g. Ludwig, 2006; Levenstein, 2006).

HSCs are typically generated from ES cells by incubating the ES cells under conditions where they form embryoid bodies and differentiate (see e.g. Fok and Zandstra, 2005; and Dang, 2004), typically in the absence of HoxA2 or HoxB2 induction. The ES cells are allowed to differentiate as embryoid bodies for approximately 2-10 days of differentiation. Then, the embryoid bodies are disaggregated to form a suspension of, cells out of which hematopoietic stem and progenitor cells are separated by sorting for CD41⁺/c-Kit⁺ double-positive cells or by sorting for CD45⁺/c-Kit⁺ double-positive cells. The separated hematopoietic cells are then cultured in a hematopoietic growth medium that induces expression of the HoxA2 or HoxB2 (e.g. hematopoietic growth medium with added doxycycline for a “Tet-on” inducible system). Various suitable hematopoietic growth media are known in the art and are commercially available (e.g. Stemline™ II Hematopoietic Stem Cell Expansion Medium; Sigma-Aldrich Corp, St. Louis, Mo.; Iscove's Modified Dulbecco's Medium (IMDM) with 10% prescreened serum; etc.)

The HSCs are cultured under conditions wherein the HoxA2 or HoxB2 is expressed, HSCs proliferate, and megakaryocyte progenitors are produced. The cell culture can be continually passaged, such as every 3, 4, 5 or 6 days for at least 30 days, and preferably at least 2, 4, or 6 months without HSC cell exhaustion. The culture medium can be supplemented with various cytokine combinations that promote the generation of megakaryocyte progenitors such as stem cell factor (SCF), interleukin (IL) 3, IL-11, and thrombopoietin (TPO) (Ahmed, 1999).

Megakaryocytes will differentiate and form platelets under conditions favorable to self-renewal of HSCs, and the platelets can be purified as desired, for example by removing the platelet-containing supernatant, or by using an apheresis device connected to the culture vessel. Optionally, when cells are passaged during culturing, a portion of the split cells can be cultured in a hematopoietic growth medium to continue HSC proliferation, and the remainder of the cells can be cultured in a differentiation medium that promotes differentiation of megakaryocyte progenitors to produce megakaryocytes and platelets. Factors that promote megakaryocyte differentiation include TPO and the Src kinase inhibitor, SU6656 (Gandhi, 2005). In preferred embodiments, the differentiation medium does not induce HoxA2 or HoxB2 expression.

For large scale propagation, the above described cell culture methods can be carried out in a platelet-producing bioreactor. The bioreactor comprises a culture vessel containing HSCs stably genetically engineered to express HoxA2 or HoxB2, and platelet-producing megakaryocyte progeny of the stem cells. In preferred embodiments, the cells are cultured in the bioreactor for at least 30 days without exhaustion of the HSCs. In various embodiments, the culture vessel has a capacity of about 0.25, 0.5, 1, 2.5, 5, 10, 25, 500 or 100 L. The bioreactor typically provides options for automated gassing, media exchange, agitation, temperature control, monitoring etc. A variety suitable cell culture bioreactors are commercially available e.g. Celligen Plus (New Brunswick Scientific Co. Inc.; Edison, N.J.) and Cellferm-pro (DasGip; Julich, Germany).

The HSCs may be grown in the bioreactor on a stromal cell layer that supports hematopoiesis. In a specific embodiment, the bioreactor culture vessel comprises an insoluble three-dimensional matrix which retains the HSCs (see e.g. Banu, 2001; and Chen, 2003). Megakaryocytes and platelets typically lift off of the cell feeder layer or matrix, making it convenient to remove and purify platelets from the medium. In a preferred embodiment, platelets are removed from the bioreactor by apheresis. The bioreactor may be connected to an apheresis device in fluid connection with the culture vessel that contains the platelet-producing megakaryocyte progeny. The apheresis device is operated to selectively remove platelets from the bioreactor. Suitable apheresis devices include the Amicus Crescendo (Baxter Biotech Corp.; Deerfield, Ill.), the MCS Plus (Haemonetics Corp.; Braintree, Mass.), and the Trima Accel (Gambro BCT; Lakewood, Colo.).

### EXAMPLE 1

### Mouse ES Cells Engineered to Inducibly Express HoxA2 Produce Platelet-Producing Progeny

HoxA2 cDNA was obtained by RT-PCR of mouse embryos (gestational age of 10.5 days) using primers based on nucleotides 31-53 (forward primer) and the complement of nucleotides 1367-1388 (reverse primer) of the mouse HoxA2 sequence (Genbank accession no NM_{—}010451.1). Mouse ES cell lines with doxycycline-inducible HoxA2 expression are made by targeting the HoxA2 cDNA into a doxycycline-inducible locus on the X-chromosome of suitably modified ES cells using methods previously described for generating a doxycycline-inducible HoxB4 cell line (Kyba, 2002).

ES cells that inducibly express HoxA2 were differentiated as embryoid bodies (EBs) by resuspending the cells (1×10⁴ cells/mL) in Embryoid Body Differentiation medium: IMDM; 15% FBS for EB; 2 mM L-alanyl-L-glutamine (Glutamax—Invitrogen); 450 μM monothioglycerol (MTG); 200 μg/ml Holo-Transferrin; penicillin/streptomycin (GIBCO); 50 μg/ml ascorbic acid. After 5 days of differentiation the EBs were disaggregated by trypsinization, and the hematopoietic progenitors separated from the rest of the cells by cell sorting for the CD41⁺/c-Kit⁺ double-positive cells. In the early embryo and in day 5 EBs, this cell surface phenotype is characteristic of the hematopoietic stem cell (CD41 is a megakaryocyte marker in the adult (Mitjavila-Garcia, 2002).

Sorted cells were plated on a layer of OP9 feeder cells (Kodama, 1994) in hematopoietic growth medium of the following composition: IMDM; 10% FBS; penicillin/streptomycin (GIBCO); 2 mM Glutamax, supplemented with rh-TPO (40 ng/ml), rm-VEGF (5 ng/ml) rh-F3L 40 ng/ml. HoxA2 was induced at this time by addition of doxycycline at 1 μg/ml. In the absence of HoxA2 induction, cells did not grow; whereas in the presence of HoxA2 expression, cell proliferation was seen. Cells were continually passaged approximately every 4 days (1×10⁵ cells were replated to a fresh layer of OP9), and cultures were kept growing in this way for up to 25 days. In some experiments, cells were switched to feeder-free conditions after the first 5 days on OP9, and grown in hematopoietic growth medium supplemented with rh-TPO (10 ng/ml) only.

At various time points, cells were evaluated by flow cytometry for lineage-specific cell-surface antigens, including CD41. Undifferentiated hematopoietic progenitor cells, including HSCs, express c-Kit, while this receptor is downregulated with differentiation. The presence of c-Kit positive cells at each passage indicated that a population of HSCs and progenitors was being sustained by HoxA2 expression.

Platelets were evaluated by centrifuging to remove cells, collecting the supernatant, which contains the platelets, fixing by addition of paraformaldehyde, and staining with CD41 antibody. Stained platelets were then analysed by flow cytometry. Mouse peripheral blood (PB) was treated in the same way and analysed as a control. CD41 positive platelets from OP9 coculture and Feeder Free (FF) culture were observed with both continual HoxA2 induction, as well as with doxycycline withdrawal. More platelets were seen with doxycycline withdrawal.

Cells were also tested for differentiation potential by replating in semisolid (methylcellulose) hematopoietic colony assay medium for myelo-erythroid or megakaryocyte progenitors. Myelo-erythroid colony assay medium was purchased from Stemcell Technologies (Vancouver, Canada), catalogue number 03434. Megakaryocyte colony assay was prepared by adding rhTPO (10 ng/mL) to cytokine-free methylcellulose (catalogue number 03234, Stemcell Technologies). Colony assays were done both with and without doxycycline to maintain HoxA2 expression. The presence of multilineage myeloid colonies (GEMM, granulocyte-erythrocyte-megakaryocyte-macrophage) is indicative of maintenance of early hematopoietic progenitors, including HSCs. GEMM colonies were observed at each passage tested. The presence of megakaryocyte colonies is indicative of the presence of megakaryocyte progenitors. Megakaryocyte colonies were observed at each passage tested. Megakaryocyte colonies were dramatically stimulated by addition of doxycycline to the colony assay to maintain expression of HoxA2.

### EXAMPLE 2

### Human HSCs Engineered to Express Hoxa2 Generate Sustained Platelet-Producing Progeny

Human HoxA2 cDNA was obtained by PCR of human genomic DNA. Protein-coding sequence from exon 1 was amplified with the following primers: HoxA2F, based on nucleotides 264-285 of the human sequence (Genbank Accession No. NM_{—}006735.3); and HoxA2M1, based on the complement of nucleotides 648-678 of the human sequence. Protein-coding sequence from exon 2 was amplified with the following primers: HoxA2M2, based on nucleotides 650-688 of the human sequence; and HoxA2R, based on the complement of nucleotides 1427-1448 of the human sequence. The two amplified products overlap by 29 base pairs. The two products were mixed and the full length ORF was produced by a second round of PCR using the primers HoxA2F and HoxA2R. The final PCR product was cloned into pGEM-Teasy (Invitrogen) which provides flanking Eco RI sites, and subcloned as an Eco RI fragment into the lentiviral vector, pSAM. pSAM is a modified version of the lentiviral vector pFUGW (Lois, 2002) in which an IRES-GFP construct has been inserted downstream of the cloning site. Thus pSAM-HoxA2 provides expression of human HoxA2 and coexpression of GFP.

Lentiviral particles are produced by cotransfecting 293T cells with pSAM-HoxA2, and packaging and envelope constructs (Lois, 2002). Viral supernatant is used to infect human CD34⁺/CD38⁻ umbilical cord blood HSCs. Infected HSCs are sorted for GFP⁺ cells by flow cytometry and grown in HSC growth medium (Stemline II, Sigma) supplemented with rhSCF, rhF3L, rhTPO, rhVEGF. Cells are passaged approximately every 4 days (1×10⁵ cells are replated into fresh medium). Flow cytometry for characterization of nucleated cells and platelets is done as in Example 1, above.

### EXAMPLE 3

### Human ES Cells Genetically Modified to Give Inducible Expression of HoxA2 Generate Sustained Platelet-Producing Progeny

The transcriptional activator for the Tet-On system, rtTA-2S-M2, (Urlinger, 2000) is cloned downstream of the Ubiquitin promoter of pFUGW, to create pFUGW-rtTA. The Ubiquitin promoter from pSAM (above) is excised and replaced with the second generation tetracycline response element, SGTRE (Agha-Mohammadi, 2004) to give pSAM-TRE. The HoxA2 gene is inserted downstream of the SGTRE and upstream of the ires-GFP to give pSAM-TRE-HoxA2. Human ES cells are coinfected with pFUGW-rtTA and pSAM-TRE-HoxA2, and cultured in the presence of doxycycline to induce HoxA2-ires-GFP expression. Cells into which both viruses integrate are green in the presence of doxycycline, and are positively selected by cell sorting, and recultured in the absence of doxycycline. Cell populations carrying both proviruses are sorted a second time to eliminate any cells that are green in the absence of doxycycline (negative selection). The cell population that is obtained by sequential positive/negative selection gives inducible expression of HoxA2 in response to doxycycline and referred to as inducible HoxA2 human ES cells.

ES cells that inducibly express HoxA2 are differentiated as embryoid bodies (EBs) by resuspending the cells (1×10⁴ cells/mL) in Embryoid Body Differentiation medium: IMDM; 15% FBS for EB; 2 mM L-alanyl-L-glutamine (Glutamax—Invitrogen); 450 μM monothioglycerol (MTG); 200 μg/ml Holo-Transferrin; penicillin/streptomycin (GIBCO); 50 μg/ml ascorbic acid. After 12-20 days, embryoid bodies are disaggregated by trypsinization and HSCs identified and purified by c-Kit/CD45 staining and flow cytometry. Inducible HoxA2 human HSCs are cultured and platelets evaluated as in Example 2, above.

The foregoing examples and detailed description are offered by way of illustration and not by way of limitation. All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings' of this invention that certain changes and modifications may be made thereto without departing from the spirit or scope of the appended claims. As used herein, the singular forms “a,” “an,” and “the,” refer to both the singular as well as plural, unless the context clearly indicates otherwise.

### REFERENCES

Agha-Mohammadi et al (2004) J. Gene Med. 6:817-828.
Ahmed et al (1999) Stem Cells 17:92-99.
Banu et al (2001) Cytokine 13:349-358.
Barde et al (2006) Mol Ther 13:382-90.
Bjornson et al (1999) Science 283:534-537.
Blesch et al (2005) Mol Ther. 11:916-25.
Brun et al (2003) Mol Ther 8:618-28.
Cardoso et al (1996) Dev Dyn. 207:47-59.
Chen et al (2003) Stem Cells 21:281-295.
Creuzet et al (2005) J. Anat. 207:447-59.
Dang et al (2004) Stem Cells 22:275-282.
Eddison et al (2004) BMC Biol. 2:14.
Fok and Zandstra (2005) Stem Cells 23:1333-1342.
Gandhi et al (2005) Blood Cells Mol Dis. 35:70-73.
Gavalas et al (2003) Development 130:5663-79.
Greer et al. (2000) Nature 403:661-665.
Janes et al (2004) J Cell Sci. 117:4157-68.
Jungbluth et al (1999) Development 126:2751-8.
Kodama et al (1994) Exp Hematol 22:979-984.
Kyba et al (2002) Cell 109:29-37.
Kyba et al (2003) PNAS100:11904-11910.
Lawrence et al (1996) Stem Cells 14:281-291.
Levenstein et al (2006) Stem Cells 24:568-574.
Logan et al (2002) Curr. Opin. Biotechnol 13:429-436.
Lois et al (2002) Science 295:868-72.
Ludwig et al (2006) Nat. Biotech 24:185-187.
Ma et al (2003) Stem Cells 21:111-117.
Markusic et al (2005) Nucleic Acids Res. 33:e63.
Mitjavia-Garcia et al. (2002) Development 129:2003-2013
Ramezani et al (2003) Blood 101:4717-4724.
Sauvageau et al. (1994) PNAS 91:12223-12227.
Shivdasani (2001) Stem Cells 19:397-407.
Thomson et al (1998) Science 282:1145-1147.
Thorsteinsdottir et al (1997) Mol Cell Biol 17:495-505.
Urlinger et al (2002) Proc Natl Acad Sci USA 97:7963-7968.
Yu (2003) Mol Ther. 7:827-38.

What is claimed is:

1. A platelet-producing bioreactor comprising a culture vessel containing (a) hematopoietic stem cells (HSCs) stably genetically engineered to express HoxA2 or HoxB2, and (b) platelet-producing megakaryocyte progeny of the HSCs.

2. The bioreactor of claim 1 wherein the HSCs are engineered with a self-inactivating lentiviral vector that expresses a HoxA2 transgene.

3. The bioreactor of claim 1 wherein the HSCs are engineered with a self-inactivating lentiviral vector that expresses a HoxB2 transgene.

4. The bioreactor of claim 1 wherein the HSCs are derived from a stem cell source selected from the group consisting of cord blood, bone marrow, and an immortalized hematopoietic stem cell line cell.

5. The bioreactor of claim 1 wherein the HSCs are stably genetically engineered to inducibly express the HoxA2 or HoxB2.

6. The bioreactor of claim 1 wherein the HSCs are the progeny of embryonic stem (ES) cells stably genetically engineered to inducibly express HoxA2 or HoxB2, and the culture vessel additionally contains a hematopoietic growth medium that induces expression of the HoxA2 or HoxB2.

7. The bioreactor of claim 1 comprising an insoluble matrix which retains the HSCs.

8. The bioreactor of claim 1 in fluid connection with an apheresis device operative to selectively remove platelets from the bioreactor.

9. A method of making platelets in the bioreactor of claim 1, the method comprising the step of: culturing the HSCs and the megakaryocyte progeny in the bioreactor to produce the platelets.

10. The method of claim 9 wherein the culturing step comprises continually passaging the HSCs for at least 30 days.

11. The method of claim 9 further comprising the step of removing the platelets from the bioreactor.

12. A method of making platelets, the method comprising the step of:
culturing hematopoietic stem cells (HSCs) to produce megakaryocyte progenitors, megakaryocytes, and platelets,
wherein the HSCs are stably genetically engineered to express HoxA2 or HoxB2, and
wherein the HSCs express the HoxA2 or HoxB2 during the culturing step.

13. The method of claim 12 wherein the HSCs are engineered with a self-inactivating lentiviral vector that expresses a HoxA2 transgene.

14. The method of claim 12 wherein the HSCs are engineered with a self-inactivating lentiviral vector that expresses a HoxB2 transgene.

15. The method of claim 12 further comprising the step of:
purifying the platelets.

16. The method of claim 12 comprising prior steps of:
stably genetically engineering embryonic stem (ES) cells to inducibly express HoxA2 or HoxB2; and
differentiating the ES cells to form the HSCs.

17. The method of claim 16 wherein the culturing step comprises:
proliferating the HSCs in a hematopoietic growth medium that induces the HSCs to express the HoxA2 or HoxB2 and produce the megakaryocyte progenitors; and
passaging the megakaryocyte progenitors to a differentiation medium that does not induce expression of the HoxA2 or HoxB2, wherein the megakaryocyte progenitors differentiate into the megakaryocytes and platelets.

18. The method of claim 12 wherein the culturing step comprises continually passaging the HSCs for at least 30 days.
